(19) United States
(12) Patent Application Publication
(10) Pub. No.: US 2002/0111298 A1
(43) Pub. Date: Aug. 15, 2002

(54) Moisturizing skin ointment - composition consisting of polymyxin B Sulfate, bacitracin zinc, neomycin (the combination of which totals 1 ounce), hydrocortisone acetate (1 ounce) and white petrolatum (13 ounces)

(76) Inventor: Joyce Ann Meeks, Hampton, VA (US)

Correspondence Address: Joyce Ann Meeks, 136 Eberly Terrace, Hampton, VA, 23669, US

(21) Appl. No.: 09/738,513

(22) Filed: Dec. 18, 2000

(51) Int. Cl. A61K 38/12; A61K 31/7036; A61K 31/7048

(52) U.S. Cl. 514/9; 514/39; 514/179; 514/28

(57) ABSTRACT

This invention relates to a moisturizing skin ointment composition consisting of polymyxin B Sulfate, bacitracin zinc, neomycin (the combination of which totals 1 ounce), hydrocortisone acetate (1 ounce) and white petrolatum (13 ounces). When combined and water evaporated, an external ointment that penetrates the skin and alleviates dry skin conditions, itching and minor scrapes and scratches is created. The properties of the antibiotic products help to promote healing of minor scrapes and scratches as well as preventing the spread of dry skin conditions. The properties of the hydrocortisone aids in the alleviation of itching associated with dry skin. Because this ointment penetrates the dermis almost immediately, the moisturizing properties of petrolatum allows the full benefit of the antibiotic products and hydrocortison to remain on/in the skin through several washings thereby alleviating the need to reapply several times a day.

### BACKGROUND OF THE INVENTION

1. Field of the Invention

This invention relates to a composition in ointment form for external use that help to soothe and alleviate dry skin conditions and symptoms.

2. Description of the Related Art

The outer layer of skin, also known as stratum corneum, is a membrane of active cells. A unique property of skin in living animals, is the process of desquamation, wherein surface cells are lost. However, these cells are replaced at the same rate as loss by the underlying cells of the skin known as the epidermal. Any alteration of this process can result in dry skin conditions such as chapping and psoriasis ranging from mild to severe.

One of the mildest forms of dry skin is a condition commonly know as “ashy” skin. This condition is more noticable among persons of darker skin tones. When left untreated, this condition causes the abnormal desquamation of skin cells and the skin becomes thicker, scaly and rough. These conditions of the skin causes the skin to itch and scratching causes the condition to spread.

This ointment was discovered for the treatment of the “ashy” skin condition. For several years before this invention, I tried to alleviate my ashy skin condition with baby oil, vaseline and lotions formulated for dry skin. I found that all had topical characteristics and were easily washed away leaving the skin in the same “ashy” state until the next application. This invention penetrates the skin and allows multiple washings before additional applications are required. Thereby providing long lasting relief of the ashy condition and allowing a normal desquamation process which leaves the skin with a natural sheen/glow that is not oily or greasy.

### SUMMARY OF THE INVENTION

This invention when applied to the skin leaves a healthy natural glow that is not oily or greasy. It soothes itching and helps prevent spreading of the dry skin condition.

### DESCRIPTION OF THE INVENTION

This invention is administered in an ointment form and may be applied to the skin on a daily basis. The formulation is as follows:

| Ingredients | Amount |
|---|---|
| White Petrolatum | 368 g (13 ounces) |
| *Neomycin | 85.4 mg |
| *Bacitracin Zinc | 9,760 units |
| *Polymyxin B Sulfate | 122,000 units |
| Hydrocortisone Acetate | 1% (hydrocortisone equivalent) (1 ounce) |

*(The combination of these ingredients equal 1 ounce)

All ingredients are combined over heat to a boiling point until separation of water and ingredients are complete approximtely twenty (20) minutes. Fragrance may be added if desired. This invention maintains an ointment form when cooled and remains so until heated.

What is claimed is:

1. A composition in form of polymyxin B Sulfate, bacitracin zinc, neomycin, hydrocortisone acetate and white petrolatum. When ingredients are combined and water evaporated, an external ointment that penetrates the skin and alleviates dry skin conditions is created.

2. A composition in form of polymyxin B Sulfate, bacitracin zinc, neomycin, hydrocortisone acetate and white petrolatum. When combined and water evaporated, an external ointment that penetrates the skin and alleviates itching associated with dry skin conditions is created.

3. A composition in form of polymyxin B Sulfate, bacitracin zinc, neomycin, hydrocortisone acetate and white petrolatum. When combined and water evaporated, an external ointment that penetrates the skin and aids in healing minor scrapes and scratches associated with dry skin conditions is created.
